(19) United States
(12) Patent Application Publication
(10) Pub. No.: US 2004/0106876 A1
(43) Pub. Date: Jun. 3, 2004

(54) Method and apparatus for acquiring and transmitting electrophysiological signals, and apparatus for recording an MRI image

(75) Inventors: Johann-Jakob Schmid, Rifferswil (CH); Roger Abacherli, Baar (CH); Jacques Felblinger, Ludres (FR)

Correspondence Address: SHOEMAKER AND MATTARE, LTD, P.O. BOX 2286, 2001 JEFFERSON DAVIS HIGHWAY, ARLINGTON, VA, 22202-0286, US

(73) Assignee: Schiller AG, Baar (CH)

(21) Appl. No.: 10/703,525

(22) Filed: Nov. 10, 2003

(30) Foreign Application Priority Data
Nov. 11, 2002 (EP) 02405968.5

(51) Int. Cl. A61B 5/04

(52) U.S. Cl. 600/509

(57) ABSTRACT

To transmit electrophysiological data from an interference region (S) of an MRI arrangement (20) to an evaluation and/or display unit (10) a signal is handled in a recording arrangement (1) arranged within the interference region (S). The ECG signal (E) is first subjected to analogue above-linear compression, is then digitized and is transmitted as a digitized signal (D) to an evaluation and/or display unit (10). The digital signal (D) is subjected to above-linear digital expansion in the evaluation and/or display unit (10).

The invention relates to a method and an apparatus for acquiring and for transmitting electrophysiological signals, particularly ECG data, and to an apparatus for recording an MRI image having the features of the precharacterizing part of the independent patent claims.

Electrocardiograms are today used in profusion for diagnosis and for monitoring patients. The ECG signals recorded by electrodes on an electrocardiograph are initially analogue signals. The signals are digitized in more modern units for the purpose of data processing and/or display. Such units are frequently operated using batteries or storage batteries so that units can be used independently of a power supply network. Converting the analogue signal into a digital signal in an A/D converter is problematical in battery-operated units as soon as relatively high input voltages arise. Such voltages can also be produced on account of interference signals, for example.

ECGs are used inter alia for monitoring patients when recording MRI images. A problem of such applications is that the MRI's strong magnetic field and radio-frequency stimuli produce interference signals in the ECG unit, for example in the electrodes, in the electrical conductors connecting the electrodes to an evaluation unit or in the evaluation unit.

Various ways have been proposed for solving these problems and for allowing patients to be monitored by means of ECG while MRI images are being recorded. Another problem is that of transmitting the recorded ECG signal without interference from the MRI tunnel to an evaluation unit.

U.S. Pat. No. 6,073,029 or U.S. Pat. No. 6,032,063 show special electrode designs and cabling configurations for recording diagnostic ECGs while an MRI is being recorded by way of example.

EP 1 050 270 shows a method for processing electrocardiographic data which is used to eliminate interference signals which are produced by an MRI.

EP 695 139 shows an ECG sensor which is suitable for use in an MRI and which is distinguished by a specific electrode design. U.S. Pat. No. 5,733,247 shows an optical connection between a patient monitor and a control desk.

WO92/21286 shows an apparatus for monitoring a patient in an MRI. To avoid interference with the MRI's magnetic field, filters and screens are used. FR 2 685 968 proposes eliminating interference between a magnetic resonance imager and an electrocardiograph, which is arranged in the imager, by using optical transmission means.

U.S. Pat. No. 4,991,580 proposes a method for data processing ECG data which are taken from a patient for whom an MRI image is being produced. For this purpose, corresponding filters and amplifiers are used.

U.S. Pat. No. 4,951,672 proposes an electrode for use in magnetic resonance imagers with specific electrical conductors.

EP 132 785 discloses the practice of screening a connecting cable arranged between the electrodes and the ECG processing unit in order to obtain an ECG signal in a magnetic resonance imager.

U.S. Pat. No. 6,052,614 likewise shows an electrocardiograph which can be used in combination with an MRI apparatus. For the purpose of transmitting the ECG data, filters and amplifiers and also optical transmission means are provided.

It is an object of the present invention to avoid the draw-backs of what is known, that is to say particularly to provide a method and an apparatus for acquiring and transmitting electrophysiological signals, such as ECG data, which method also allows the acquired signal to be digitized during battery operation. Another object of the invention is to provide a method and an apparatus for acquiring and transmitting signals which allow signals to be acquired at the same time as an MRI image is being recorded. Another object of the invention is to provide an MRI apparatus which allows electrophysiological signals to be acquired at the same time.

The invention achieves these objects with a method and with an apparatus having the features of the characterizing part of the independent patent claims.

The inventive method can be applied to various types of electrophysiological signals, for example electroencephalograms or electrocardiograms. In the present case, the invention is explained with reference to an ECG recording arrangement. It goes without saying that the invention can be used in a similar manner in combination with other apparatuses for measuring other electrophysiological signals.

The inventive method involves ECG data being acquired in an ECG recording arrangement. In this case, an analogue ECG signal is produced. The ECG signal is then compressed. This produces a compressed analogue signal. Compression takes place in above-linear fashion. Here and below, above-linear is understood to mean a compression function in which compression takes place at a greater than linear level. Above-linear thus means that the degree of compression at relatively high values of the output signal is greater than at relatively low values of the output signal. The compression can typically be polynomial.

The compressed analogue signal is then converted into a digital signal. The digital signal is transmitted from the ECG recording arrangement to an evaluation and/or display unit. The evaluation and/or display unit expands the transmitted digital signal again in line with the compression function. The expanded digital signal is processed further or displayed in the evaluation and/or display unit.

It is also possible for the digital signal to be processed digitally in the ECG recording arrangement. By way of example, the signal could be digitally filtered.

The above-linear compression of the analogue ECG signal allows interference signals having high amplitudes to be reduced, in particular. The compressed analogue signal can easily be converted into a digital signal in an A/D converter without the need for high voltages from a voltage source.

It is a particularly simple matter for the analogue ECG signal to be compressed logarithmically and accordingly for the digital signal to be exponentially expanded again digitally.

The inventive method for acquiring and transmitting ECG data can be applied particularly advantageously at the same time as MRI images are being recorded. In this case, the ECG recording arrangement is arranged within an electromagnetic interference region of the MRI arrangement. The evaluation and/or display unit is arranged outside this interference region. The local conversion of the ECG data into a digital signal allows the ECG data to be sent easily in digitized form from the MRI tunnel to an external evaluation unit. The inventive apparatus also allows the ECG recording arrangement to be operated with batteries, so that no supply of current from external voltage or current sources is necessary.

The inventive method can be used both in closed and in open MRI systems.

In line with one preferred embodiment, the ECG signal is compressed and preferably also the compressed analogue signal is converted into a digital signal in a processor which is mounted directly on an electrode of the ECG recording arrangement. This makes it possible to prevent, in particular, interference signals which could be produced in connecting lines between the electrodes and evaluation units.

In line with another preferred embodiment, the digital signal is transmitted from the ECG recording arrangement to the display and/or evaluation unit optically. Optical transmission of the digitized signal is advantageous particularly on account of the insusceptibility to the production of interference signals.

Alternatively, other transmissions, particularly transmission by electrical conductors, radio or else mechanical transmission means (for example pneumatically), are conceivable.

The inventive method can also be used, in particular, for the purpose of using an electrophysiological signal acquired while an MRI is being recorded, for example an ECG signal, for the purpose of “gating”. That is to say that information in the ECG signal can be used to trigger or synchronize an MRI. This is possible, in particular, because the movement of the heart is known precisely as a function of time in the case of ECG signals, for example. Alternatively, the signal can be used to monitor patients.

As an alternative, it is also conceivable for the digitized signal to be transmitted electrically via a screened connecting line or electromagnetically by radio, e.g. via Bluetooth.

The inventive apparatus is used for acquiring and for transmitting electrophysiological signals, particularly ECG data. The apparatus is particularly suitable for carrying out one of the methods cited above. The apparatus has an inherently conventional recording arrangement for producing at least one electrophysiological signal, particularly an ECG signal.

The apparatus also contains an evaluation and/or display unit. The evaluation and/or display unit is used for handling and/or for displaying ECG data. The apparatus is provided with transmission means which are used for transmitting data from the ECG recording arrangement to the display and/or evaluation unit. In line with the invention, the ECG recording arrangement has means for above-linear compression of the ECG signal and for forming a compressed analogue signal. An A/D converter can be used to convert the compressed analogue signal into a digital signal. The display and/or evaluation unit also contains means for digitally expanding the digital signal. The digital expansion takes place in above-linear fashion in accordance with the previous compression. The expansion function is a reciprocal function of the preceding compression function.

Preferably, the means for compressing the ECG signal are designed for logarithmically compressing the ECG signal, and the means for digital expansion are designed for exponentially expanding the digital signal.

In line with one preferred exemplary embodiment, the means for compression and preferably also the A/D converter are formed by a computer arrangement which is mounted directly on an electrode of the ECG recording arrangement. It is also conceivable for a corresponding computer arrangement to be provided on a plurality of electrodes.

The inventive apparatus can be used to reduce the power consumption, since the input signals into the A/D converter have relatively low voltages.

Another preference is for optical transmission means to be transmitted for transmitting the digitized signal from the ECG recording arrangement to the display and evaluation unit. Other, particularly electrical, transmissions, particularly by means of a screened cable or radio, are alternatively conceivable. In principle, mechanical transmissions, for example pneumatic transmission of the signal, are also conceivable and possible.

The inventive apparatus for recording an MRI is essentially of conventional design. In addition, the apparatus is produced with an apparatus as described above for acquiring electrophysiological data.

The invention is explained in more detail below in exemplary embodiments and with reference to the drawings, in which:

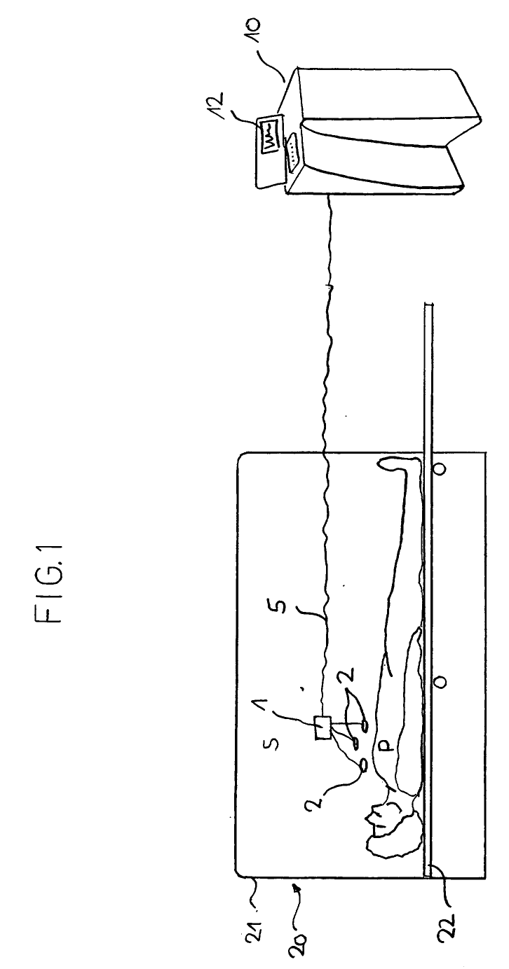

FIG. 1 shows a schematic illustration of an inventive apparatus for simultaneously recording an MRI image and an ECG,

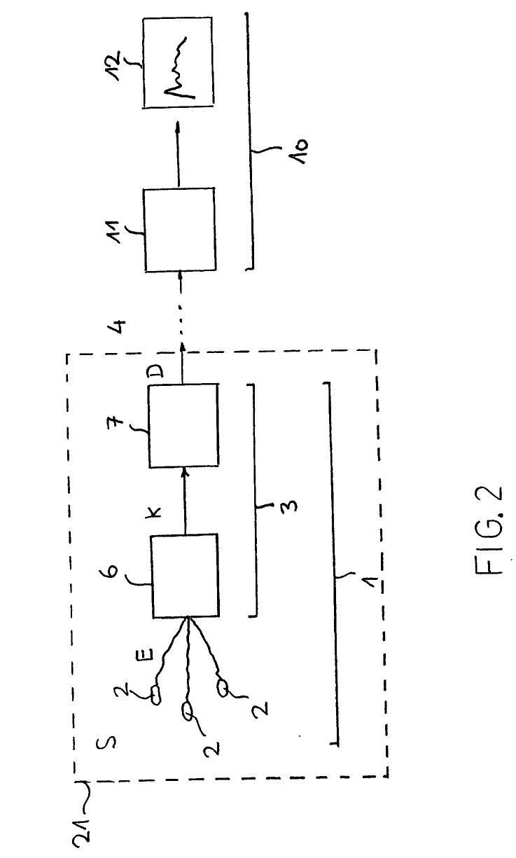

FIG. 2 shows a schematic illustration of individual parts of the inventive apparatus,

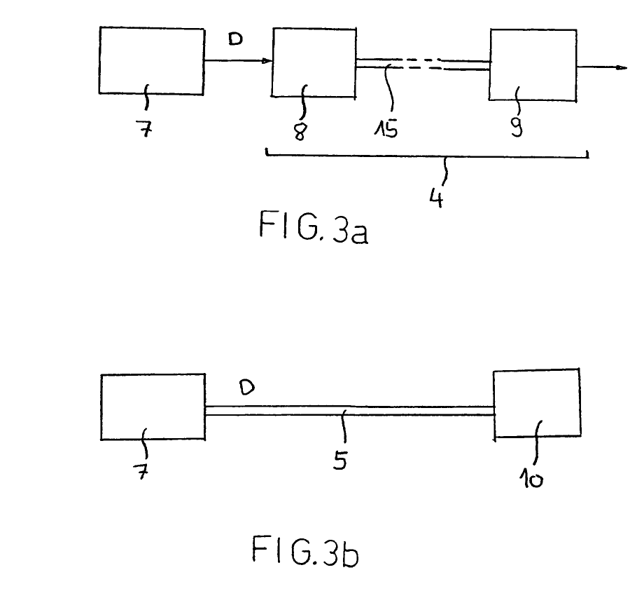

FIG. 3a shows a schematic illustration of a first embodiment of transmission means,

FIG. 3b shows a schematic illustration of a second embodiment of transmission means, and

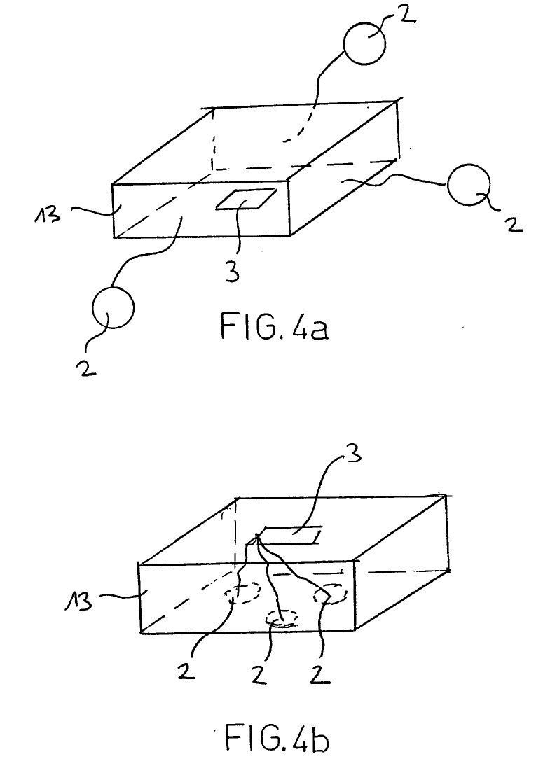

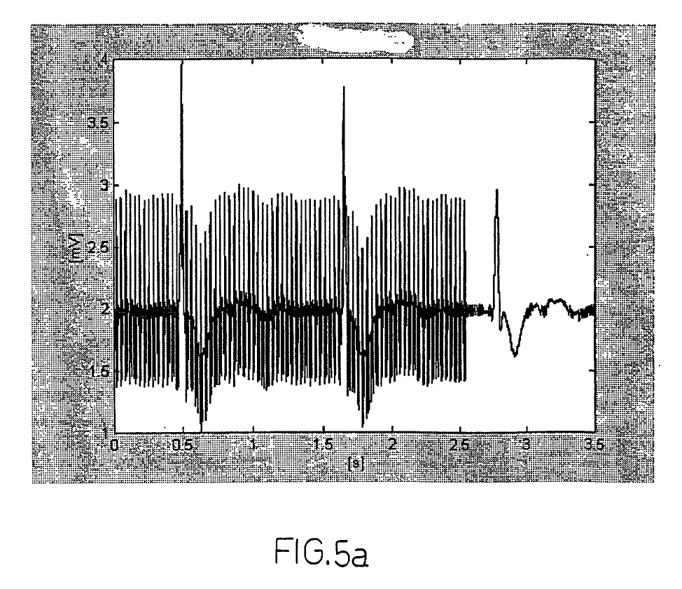

FIGS. 4a, 4b show schematic illustrations of computer arrangements connected to electrodes, and

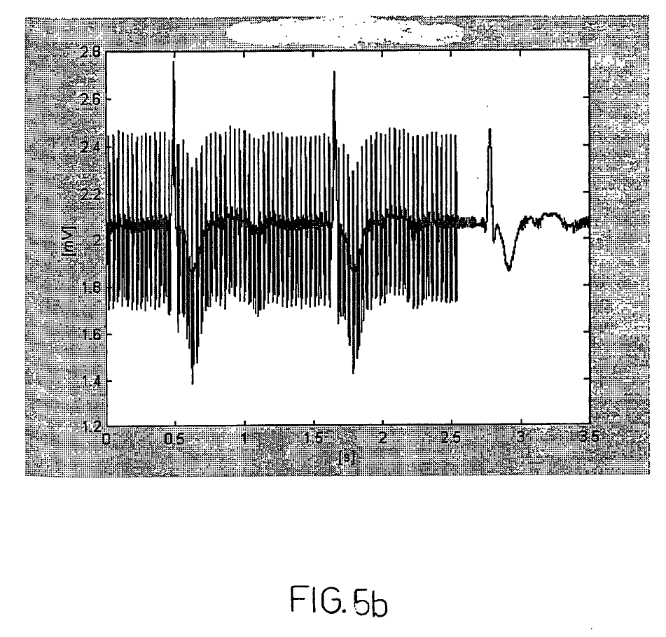

FIGS. 5a, 5b show a schematic illustration of an uncompressed and of a compressed ECG signal with interference.

FIG. 1 shows an MRI arrangement 20. The arrangement 20 has, conventionally, components such as a magnetic coil, a transmitter and a receiver and corresponding detection means for producing MRI images, these components not being shown in more detail. The MRI arrangement 20 has a tunnel. The inventive apparatus and the inventive method can alternatively be used in conjunction with open MRI systems. For the purpose of diagnosis, a patient P can be pushed into the tunnel on a displaceable bed 22. The MRI arrangement 20 is screened from electromagnetic waves by a screen 21 which forms a Faraday cage. The specific design of the MRI arrangement 20 is not the subject matter of the present invention. It is not described in more detail.

The MRI arrangement 20 has an ECG recording arrangement 1. In the exemplary embodiment, the ECG recording arrangement 1 is provided with three electrodes 2. It is also possible to use more or fewer electrodes. The ECG recording arrangement 1 has, conventionally, corresponding inputs for the electrodes, a microprocessor for processing the ascertained ECG signals and a power supply. The ECG recording arrangement 1 is also provided with an output connection for transmitting data. FIG. 1 shows a screened cable 5 for transmitting the data, said cable transmitting ECG data to an evaluation and/or display unit 10 situated outside the MRI arrangement 20. The evaluation and/or display unit 10 is provided with a display 12 for displaying ECG data. The evaluation and/or display unit 10 is situated outside an interference region S of the MRI arrangement. The ECG recording arrangement 1 is situated in the electromagnetic interference region S bounded by the screen 21.

FIG. 2 schematically shows individual components of the inventive apparatus. Besides elements of the MRI arrangement 20, which are not shown in detail, the screen 21 contains an ECG recording arrangement 1. The ECG recording arrangement 1 has three electrodes 2 which can be used to produce an analogue ECG signal E. The electrodes 2 are connected to a computer unit 3 by means of cables which are not denoted in more detail. First, the analogue ECG signal E is compressed in compression means 6 to form a compressed analogue signal K. The compressed analogue signal K is digitized in an A/D converter 7 to form a digital signal D. The compression means 6 are formed by a conventional compressor, e.g. by a logarithmizer.

The A/D converter used is a conventional 16-bit low-power A/D converter, for example.

It is also conceivable to use a signal processor to perform additional processing operations on the digital signal, for example to filter the signal.

In the exemplary embodiment shown in FIG. 2, the digital signal D is transmitted from the screen 21 to the evaluation and/or display unit 10 using optical transmission means 4. The evaluation and/or display unit 10 contains means 11 for digitally expanding the digital signal D. The means for expansion are formed by an appropriately programmed microprocessor, for example. The expanded digital signal is processed in the evaluation and/or display unit 10 in a manner which is known per se and is displayed on a display 12. The expanded digital signal can be processed conventionally in order to eliminate interference by the MRI arrangement 20. This can be done by means of digital filtering or template matching, for example.

FIG. 3a schematically shows one possible embodiment of optical transmission means. The signal D digitized in the A/D converter is sent to an optical receiver 9, typically a photodetector, via an optical fibre 15 using an optical transmission arrangement 8, for example a laser diode with appropriate data conditioning, and said optical receiver converts the optical signal back into an electrical signal and forwards it to the means for expansion 11.

FIG. 3b shows an alternative with a screened cable 5 for transmitting the digital signal D.

The components of the inventive apparatus which are arranged within the interference region 2, particularly the compression means, the A/D converter and any components of optical transmission means, are screened from electromagnetic interference in a manner which is known per se. These components can be arranged in a screened housing. To prevent interference in the connecting lines between the electrodes 2 and the computer unit 3, it is also conceivable in accordance with the invention for the computer unit 3 to be arranged directly on one or more electrodes.

FIG. 4a shows a first exemplary embodiment of an apparatus in accordance with the invention. The computer unit 3 is arranged in a housing 13. Three electrodes 2 are provided on the housing 13 via short electrical connections. The housing 13 is formed from copper, for example, or from another antimagnetic material. The housing can typically have a size of a few centimetres.

FIG. 4b shows an alternative embodiment. In the embodiment shown in FIG. 4b, the computer unit 3 is likewise arranged in a housing 13, preferably made of copper. The electrodes 2 are integrated directly in the housing 13. This ensures that no interference occurs in the connecting lines between the electrodes 2 and the computer unit 3 either.

FIGS. 5a and 5b show typical ECG signals and their processing.

FIG. 5a shows a conventional analogue ECG signal. Interference produced by an MRI arrangement is shown in the ECG signal.

FIG. 5b shows the logarithmically compressed analogue signal K. Compression takes place in accordance with the following formula:

K=a×log(b×E).

The factors a and b can be chosen in a suitable manner, with the optimum factors being able to be found by experiment, according to the initial situation. The calculation is performed using a natural logarithm.

The uncompressed signal was recorded under the influence of a strong electromagnetic gradient field. The magnetic field is turned on between the time 0 and 2.5 seconds. The magnetic field results in interference of approximately 3 mV. This interference is overlaid on the QRS complex, so that maxima of up to approximately 4 mV arise. This amplitude is too great for signal processing with the desired units, particularly an operational amplifier and an A/D converter.

FIG. 4b shows the signal following compression. The interference has been reduced to 2.4 mV. The logarithmic compression means that the amplitude of the maximum signal is now just 2.8 mV. This signal can be processed further without difficulty.

The digital signal D is expanded in the expansion unit as follows:

Dₑ=1/b×e^{(D/a)}

1. A method for acquiring electro-physiological data in a recording arrangement and for transmitting the data to an evaluation and/or display unit, said method comprising steps of:
acquiring a signal in the recording arrangement, above-linear compressing of the signal in order to form a compressed analogue signal,
converting the compressed analogue signal into a digital signal,
transmitting the digital signal to the evaluation and/or display unit, and
expanding the digital signal digitally into a expanded digital signal,
displaying and/or processing the expanded digital signal.

2. A method according to claim 1, wherein the signal is compressed logarithmically.

3. A method according to claim 1, wherein the electrophysiological data are acquired and transmitted at the same time that an MRI diagnosis is being performed on a patient, with the recording arrangement being situated within and the evaluation and/or display unit being situated outside an electromagnetic interference region of an MRI arrangement for performing the diagnosis.

4. A method according to claim 1, wherein the analogue signal is converted into the digital signal in a processor which is mounted directly on an electrode of the recording arrangement.

5. A method according to claim 1, wherein the digital signal is transmitted to the evaluation and/or display unit optically.

6. A method according to claim 1, wherein the digital signal is transmitted to the evaluation and/or display unit electrically via a shielded connecting line.

7. A method according to claim 1, wherein the digital signal is transmitted to the evaluation and/or display unit wirelessly by radio.

8. An apparatus for acquiring and transmitting electrophysiological data, in a method according to claim 1, said apparatus comprising:
a recording arrangement for producing at least one signal,
an evaluation and/or display unit for treating and/or displaying electrophysiological data,
transmission means for transmitting data from the recording arrangement to the evaluation and/or display unit,
the recording arrangement having means for above-linear compression of the signal for forming a compressed analogue signal and an A/D converter for converting the compressed analogue signal into a digital signal, and
the evaluation and/or display unit has means for digitally expanding the digital signal.

9. An apparatus according to claim 8, wherein the means for compressing the signal logarithmically compresses the signal.

10. An apparatus according to claim 8, wherein the means for compression are formed by a computer arrangement arranged directly on an electrode of the recording arrangement.

11. An apparatus according to claim 10, wherein the A/D converter is also formed in the computer arrangement arranged directly on an electrode of the recording arrangement

12. An apparatus according to claim 8, wherein the transmission means are optical transmission means.

13. An apparatus according to claim 8, wherein the transmission means are formed by a shielded cable.

14. An apparatus for performing an MRI diagnosis in combination with an apparatus for simultaneously acquiring electro-physiological data according to claim 8.
